(19) United States
(12) Patent Application Publication
(10) Pub. No.: US 2006/0167475 A1
(43) Pub. Date: Jul. 27, 2006

(54) Instrument for the surgical closure of a trocar puncture

(76) Inventors: Georg Bischof, Wien (AT); Thomas Wild, Wien (AT); Ewald Unger, Wien (AT)

Correspondence Address: WENDEROTH, LIND & PONACK, L.L.P., 2033 K STREET N. W., SUITE 800, WASHINGTON, DC, 20006-1021, US

(21) Appl. No.: 11/336,820

(22) Filed: Jan. 23, 2006

(30) Foreign Application Priority Data
Jan. 24, 2005 (AT) A 95/2005

(51) Int. Cl. A61B 17/04

(52) U.S. Cl. 606/144

(57) ABSTRACT

An instrument for the surgical closure of a trocar puncture through a posterior fascia introduced during a laparoscopic operation comprises

a tube (1) with a forward insertion end (2) and a rearward end (3), in whose shell wall (5) opposing window openings (7) are formed, in which terminates at its margins remote from the insertion end (2) of the tube (1) at least one bore (8) extending in the shell wall (5) in the longitudinal direction of the tube (1),

hollow needles (10), each disposed in one of the bores (8) and displaceable between a retracted position, in which they clear the particular window opening (7), and an end position, in which they cross the window opening (7), and each having an inner channel (17),

wherein each of the inner channels (17) of the hollow needles (10) forms a section of a guide channel for guiding through a suture thread (36), and this guide channel further includes at least one curved connecting section (18), which connects the front ends of the inner channels (17) of two hollow needles (10) when they are in their end positions and crossing opposing window openings (7), which, over its entire longitudinal length, is covered with respect to the outer space encompassing the tube (1) by an openable closure device (23) or is connected via a slot with the outer space encompassing the tube (1), wherein a segment of the suture thread (36) located in the connecting section (18) can be brought out of the connecting section (18) of the guide channel into the outer space through the slot or after opening the closure device (23).

### BACKGROUND OF THE INVENTION

a) Field of the Invention

The invention relates to an instrument for the surgical closure of a trocar puncture through a posterior fascia introduced during a laparoscopic operation.

b) Description of Related Prior Art

To perform laparoscopic operations, trocars are introduced into the abdominal cavity via punctures through the abdominal wall. The abdominal cavity is lined on the inside by the peritoneum. Toward the outside follows a fascia, which is referred to as posterior fascia and which is of essential significance for the stability of the abdominal wall. A muscle layer follows in the direction toward the outside, which, again, is followed by a fascia, a layer of fat and lastly the skin.

Starting at a diameter of the trocar of 10 mm, the posterior fascia should be closed when a trocar is pulled out of the abdominal cavity at the end of the operation in order to prevent puncture canal hernias. Since the puncture canal collapses after the trocar has been pulled out, for this purpose the tissue must be pulled apart with forceps and the needle must be inserted with a needle holder into the area of the posterior fascia. For the purpose of applying a suture, the tissue must be pierced accordingly. Due to lack of visibility, this procedure is also difficult to perform and becomes more difficult the thicker the abdominal wall of the patient.

To facilitate the application of a fascial suture, an auxiliary instrument is already known which is formed by a cone comprising two bores extending obliquely with respect to the longitudinal direction and crossing one another. The cone is introduced into the puncture canal until the entrances into the two bores lie outside of the abdominal wall and the exits of the bores are located at the sites at which the needles are intended to pass through the tissue. The needle with the suture thread disposed thereon is guided through one of the bores and, at the exit of the bore, passed through the tissue into the abdominal cavity. The thread is released from the needle. The needle is pushed through the second bore and the thread is inserted into the needle with a carrier, which is introduced through another thinner trocar, whereupon the needle is again retracted. Of disadvantage is here, inter alia, that during this procedure the pneumoperitoneum must be maintained. In order to check the procedure, the optical system must be transferred onto a thinner trocar (which does not require closure of the fascia). While the needle is pushed into the abdominal cavity, there is also the risk of injury to the patient.

Due to the existing difficulties, in practice closure of the fascia is frequently not carried out, with the risks for the patients entailed therein.

### AIM AND SUMMARY OF THE INVENTION

The aim of the invention is to provide an improved instrument of the above described type with which a fascial suture thread can simply and safely be placed in position.

According to the invention this is attained with an instrument for the surgical closure of a trocar puncture through a posterior fascia introduced in a laparoscopic operation comprising:
a tube with a forward insertion end and a rearward end, in whose shell wall opposing window openings are formed in which terminates at its margins remote from the insertion end of the tube at least one bore extending in the shell wall in the longitudinal direction of the tube,
hollow needles, each disposed in one of the bores and displaceable between a retracted position, in which they clear the particular window opening, and an end position, in which they cross the window opening, and each having an inner channel,
wherein each of the inner channels of the hollow needles forms a section of a guide channel for guiding through a suture thread, and this guide channel further includes at least one curved connecting section, which connects the front ends of the inner channels of two hollow needles when they are in their end positions and crossing opposing window openings, which, over its entire longitudinal length, is covered with respect to the outer space encompassing the tube by an openable closure device or is connected via a slot with the outer space encompassing the tube, wherein a segment of the suture thread located in the connecting section can be brought out of the connecting section of the guide channel into the outer space through the slot or after opening the closure device.

In order to apply a fascial suture with an instrument according to the invention, the tube is placed with its forward insertion end forward into the puncture canal of the trocar to such depth that the window openings in the shell wall are located at that site at which the tissue must be pierced. Due to its intrinsic elasticity, the tissue projects slightly into the tube in the area of the window openings. If necessary, this can also be enhanced by generating an underpressure in the tube or by mechanically pressing or pulling (for example manually from the outside or from the inside by means of forceps) the tissue into the tube. The hollow needles are subsequently displaced from their retracted positions, in which they clear the window openings, into their end positions, in which they cross the window openings. The tissue protruding into the window openings is pierced in the process. In the advanced end positions of the hollow needles a guide channel is formed for the passage of a threader element, which guide channel extends through the inner channels of the hollow needles and through the curved connecting section connecting the front ends of the inner channels of the hollow needles.

After the thread has been pulled into the guide channel by means of the threader element and runs through it, the hollow needles are slid back into their retracted positions in which they clear the window openings. The tube can now be pulled out of the puncture channel, and the segment of the thread, located in the connecting section of the guide channel, is brought out of it transversely to the particular local longitudinal extent of the connecting section. For this purpose, the guide channel in the connecting section is for example delimited with respect to the outer space encompassing the tube by a tearable strip with a correspondingly thin wall thickness. When the tube is pulled back, the segments of thread located in the hollow needles are pulled out of them (through the terminations of the inner channels located at the margins of the window openings of the hollow needles). The suture threads, now extending at both sides of the puncture channel of the trocar through the posterior fascia to be closed, can subsequently be tied to form the suture.

It is also conceivable and possible that the connecting section of the guide channel is connected over its entire length through a slot with the outer space encompassing the tube. In this case the threader element must have a thickness greater than the width of the slot in order to ensure its guidance through the connecting section of the guide channel. However, a closure device for the connecting section of the guide channel is preferably provided.

In a feasible embodiment of the invention the tube is closed at its forward insertion end. The connecting section can in this case extend through the forward front side of the frontal face of the front wall forming the tube.

In another feasible embodiment of the invention the tube can be formed such that it is open at its insertion end, the instrument being simultaneously developed as a trocar. The curved connecting section of the guide channel can in this case extend in the shell wall of the tube into the area in front of the window openings.

The connecting section of the guide channel is preferably formed by a groove introduced on the outside of the tube, which groove is initially closed by a closure device. In the forward end position of the hollow needles a continuous guide channel closed over its entire length can consequently be formed. To bring the thread out of the connecting section, the closure device is subsequently opened. This can be accomplished for example by tearing a strip-form film covering the groove in the tube and fastened (in particular by adhesion) on both sides of the groove, the film forming in this case the closure device. It would also be conceivable and possible, that a strip-form film covering the groove in the tube is only adhered on the tube on one side of the groove with such adherency that the film, when the tube is pulled out of the puncture channel, can be torn off on one side with the thread thus being released from the groove.

A displacement device is advantageously provided for the simultaneous displacement of the hollow needles between their retracted positions clearing the window openings and their end positions crossing the window openings. For this purpose in an advantageous embodiment of the invention a rotatable actuating annulus can be provided, which, during its rotation, displaces via a slotted link guidance a needle mounting annulus mounting the hollow needles in the longitudinal direction of the tube.

In the following further advantages and details of the invention will be explained in conjunction with the attached drawings, with further aims of the invention becoming evident.

### BRIEF DESCRIPTION OF THE DRAWINGS

In the drawing depict:

FIG. 1 an oblique view of an instrument according to the invention according to a first embodiment example of the invention in the retracted positions of the hollow needles,

FIG. 2 the instrument of FIG. 1, parts of the instrument shown taken apart in the manner of an exploded view,

FIG. 3 an enlarged depiction of the closure device for the connecting section of the guide channel, viewed obliquely

FIG. 4 an enlarged depiction of the front end section of the instrument viewed obliquely,

FIG. 5 a depiction analogous to FIG. 4, however without the closure device of the connecting section of the guide channel,

FIG. 6 a depiction corresponding to FIG. 5, however in the advanced end position of the hollow needles,

FIG. 7 a longitudinal section through the instrument in the retracted position of the hollow needles, with the tissue layers of the abdominal wall indicated schematically,

FIG. 8 a depiction corresponding to FIG. 7, however in an intermediate position of the hollow needles and with an endoscope introduced into the tube,

FIG. 9 a depiction corresponding to FIG. 7, however in the advanced end position of the hollow needles,

FIG. 10 an enlarged detail of FIG. 9,

FIG. 11 a depiction corresponding to FIG. 9, however with a threader element pushed through the guide channel and a suture thread attached thereto,

FIG. 12 a depiction corresponding to FIG. 11, however with the thread threaded through the guide channel,

FIG. 13 the front section of the instrument viewed obliquely and the abdominal wall in schematic section after the tube has been pulled from the puncture channel,

FIG. 14 an oblique view of a two-part embodiment of a closure device for the connecting section of the guide channel,

FIG. 15 an exploded view of a further embodiment of an instrument according to the invention,

FIG. 16 an enlarged depiction of the closure element for the connecting section of the guide channel viewed obliquely,

FIG. 17 a front end section of the instrument according to this further embodiment in the advanced end position of the hollow needles, without the closure device,

FIG. 18 an exploded view of yet a further embodiment of an instrument according to the invention,

FIG. 19 and FIG. 20 depictions of the end section of the instrument according to this embodiment in two positions of the cutting tool,

FIG. 21 a schematic oblique view of a front end section according to yet a further embodiment of an instrument according to the invention,

FIG. 22 a block diagram of the electronic analysis unit,

FIG. 23 a schematic depiction of a feasible embodiment of a countersupport for the particular hollow needle, and

FIG. 24 a schematic depiction of a further embodiment of a hollow needle.

### DESCRIPTION OF THE PREFERRED EMBODIMENT EXAMPLE

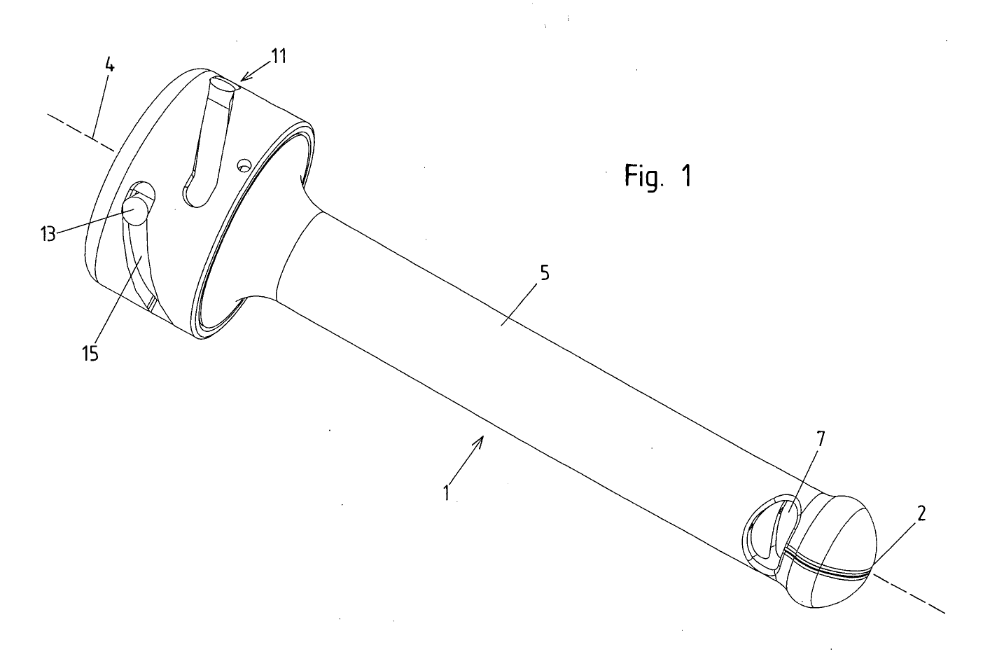

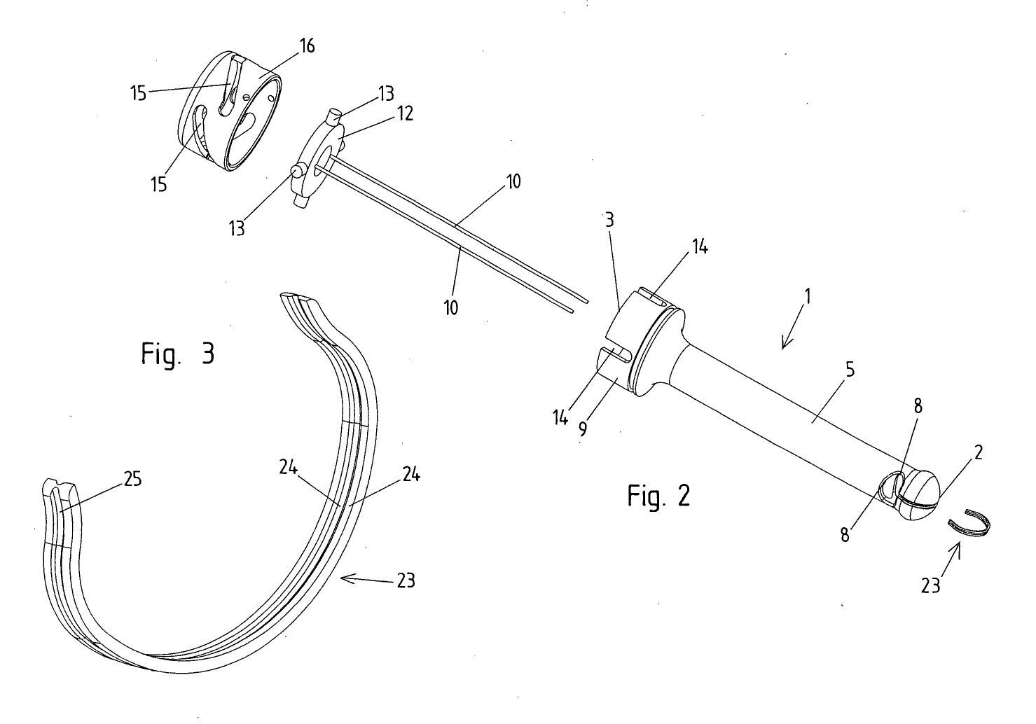

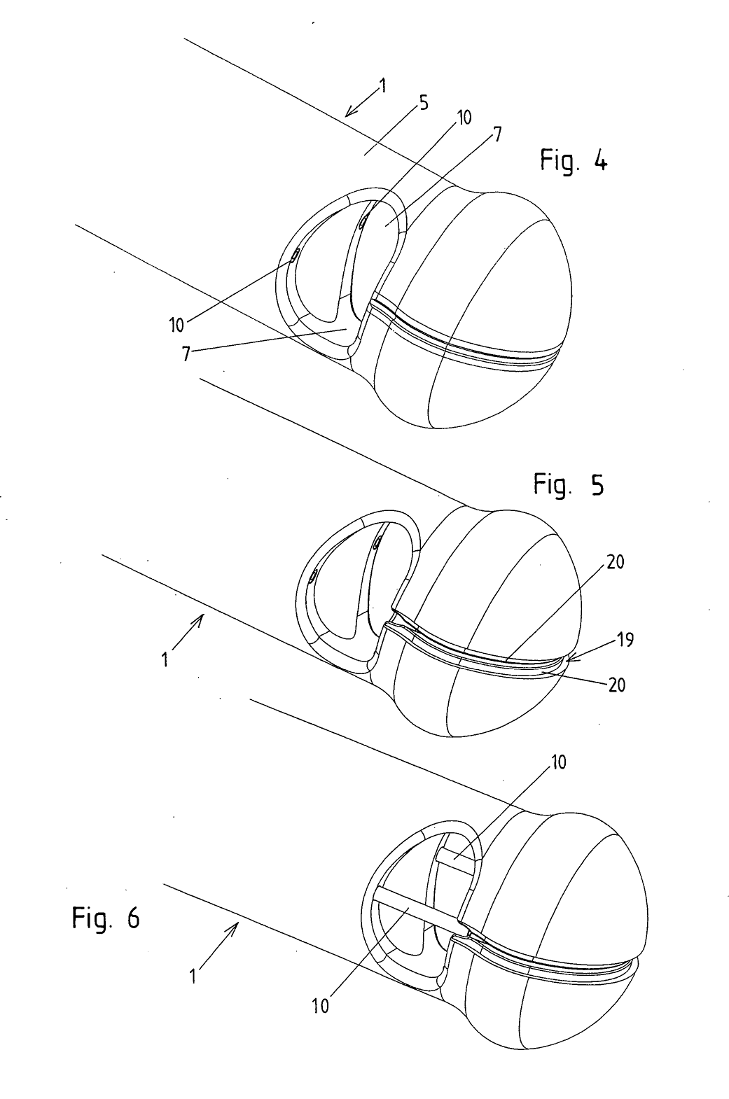

A first embodiment of an instrument according to the invention is depicted in FIG. 1 to 13. The instrument comprises a tube 1 (=a sleeve) consisting preferably of synthetic material, such as is utilized for example for trocars, and a forward insertion end 2 and a rearward end 3. When utilizing the instrument, the tube 1 is to be introduced with the insertion end forward into a puncture channel of a trocar.

If, within the scope of this document, the terms ‘forward or front’ and ‘rearward or rear’ are used, they are intended to indicate the position with respect to the insertion end 2, i.e. a part located further forward is closer to the insertion end than a part located further rearward.

The tube 1 has a shell wall 5, which, viewed in cross section, is annular and which encompasses an inner hollow space 6 extending in the longitudinal direction of the tube. The tube 1 is open at its rearward end 3. In this embodiment example the tube 1 is closed at its forward insertion end.

In the shell wall 5 of tube 1 two opposing window openings 7 are formed through which the inner hollow space 6 of the tube is connected with the outer space encompassing the tube 1. The lateral window openings 7 are consequently located transversely to the longitudinal axis 4 of tube 1.

The opposing window openings 7 are preferably located in a front end section of the tube, best at least in the front fourth of the longitudinal extent of tube 1.

Preferably at least 30% of the circumference of the tube is exposed in each instance by the window openings 7 (at the widest site of the particular window opening). In the longitudinal direction of the tube the size of the particular window opening at its widest site is best at least 10 mm. A range from 10 to 30 mm is preferred.

In the shell wall 5 of the tube 1 bores 8 located parallel to the longitudinal axis 4 of tube 1 extend in the longitudinal direction of the tube. In this embodiment example in each particular window opening 7 terminates one bore 8, and specifically at the margin of the window opening 7 remote from the forward insertion end 2.

Starting from the window openings 7 in the shell wall 5 the bores 8 extend in the rearward direction in the shell wall 5 and specifically over the entire length of that section of tube 1 in which it has a constant inner diameter.

The rearward end of this section is adjoined in this embodiment example by a section 9 with an increased inner and also outer diameter. The bores 8 terminate at the rearwardly directed step between these two sections of tube 1.

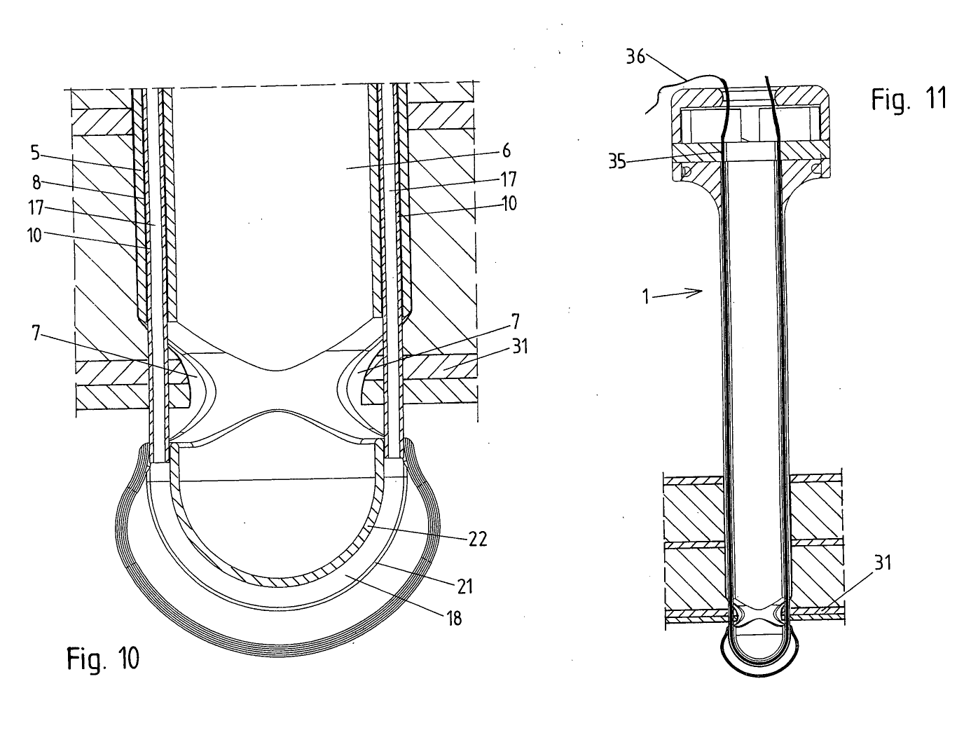

In each of the bores 8 is displaceably guided a hollow needle 10. The hollow needles 10 are herein displaceable between a retracted position (cf. for example FIGS. 1, 4, and 7), clearing the particular window opening 7, and an advanced end position (cf. for example FIG. 6, FIG. 9 and FIG. 10) crossing the particular window opening 7. In their advanced end positions as well as also in their retracted positions clearing the window openings 7 the hollow needles 10 are located in bores 8 over a portion of their longitudinal extents. In their retracted position the front ends of the hollow needles 10 do not project beyond the bores 8.

In this embodiment example the hollow needles 10 are displaceable between their retracted position and their advanced end position by means of a slotted link guidance 11. For this purpose the hollow needles 10, depending on the displacement position projecting with a rearward end section more or less far from the bores 8, are disposed at their rearward ends on a needle mounting annulus 12. The needle mounting annulus 12 comprises for this purpose bores extending in the axial direction, into which the ends of the hollow needles 10 are inserted and in which they are fastened, for example by adhesion.

The needle mounting annulus 12 is located in section 9 of tube 1 and includes outwardly projecting slotted link pegs 13, which penetrate through slots 14 disposed in section 9 and extending in the axial direction of tube 1. The slotted link pegs 13 engage into slotted links 15, which are disposed in the actuating annulus 16 encompassing section 9. By rotating the actuating annulus 16, the slotted link pegs 13 are displaced by the obliquely extending slotted links 15 in the axial direction (the needle mounting annulus 12 being secured against rotation through the slots 14), wherein the needle mounting annulus 12, and with it the hollow needles 10, are displaced in the axial direction of tube 1.

Displacement devices for the simultaneous displacement of the two hollow needles 10 in the axial direction can also be formed differently, for example via cam disks, toggle mechanisms or spindle threaded gear pairs (=threaded transmissions).

In the advanced end position of the hollow needles 10, in which they cross the window openings 7 (cf. FIGS. 6, 9 and 10), a continuous guide channel for guiding through a thread is formed, as will be explained later in greater detail. Each of the inner channels 17 of the hollow needles 10 forms a section of this guide channel. The guide channel comprises further a curved connecting section 18, which connects the front ends of the inner channels 17 of the hollow needles 10 when they are in their front end positions.

In this embodiment example for the formation of this connecting section 18 extends a groove 19 over the front wall closing the tube 1 at the front, which groove extends up to the two opposing window openings and terminates in them.

The groove 19, which in this embodiment example is relatively deep, viewed over the depth of the groove 19 has a wider section adjoining the outer surface of tube 1, which is adjoined via a step 20 by a section with a decreased groove width, which is adjoined via a step 21 by a section with, again, a wider groove width, which forms the connecting section 18. The connecting section 18 of the guide channel is delimited toward the inner hollow space 6 of tube 1 by a partitioning wall 22, whose surface delimiting the connecting section 18 forms the groove base of groove 19.

In the widened section of groove 19 adjoining the outer surface of tube 1 is disposed a closure device 23, which covers the connecting section 18 over its entire longitudinal extent and consequently closes it toward the outer space encompassing the tube 1. This closure device is here formed by a film-like strip shown at an enlarged scale in FIG. 3 and formed of a synthetic material. The strip has over its entire longitudinal extent thicker regions extending on both sides and an intermediate thinner region, which forms a type of rated break point of the strip, as will be explained later in further detail. With its thicker regions the strip is adhered on steps 20.

In the depicted embodiment example tube 1 includes, adjoining the window openings 7 toward the insertion end 2, an increased head which forms the insertion end 2 or the forward frontal face of the tube 1. It would also be conceivable and possible for the tube 1 to retain the same outer diameter up to its rounded-off end or the end formed in the shape of a cone with a rounded-off tip.

Completing a fascial closure by applying a suture by means of an instrument according to the invention will be explained in the following with reference to FIG. 7 to 13. Tube 1 is first inserted into the puncture channel in the abdominal wall 26, as is shown in FIG. 7. The structure of the abdominal wall 26 is depicted schematically. The skin 27 is adjoined by the fatty layer 28 delimited by a fascia 29 against the subjacent muscle layer 30. Beneath the muscle layer 30 is located the posterior fascia 31, which is of considerable importance for the stability of the abdominal wall. Below the fascia 31 is located the peritoneum 32, which lines the abdominal cavity 33.

Tube 1 is inserted into the puncture channel to such depth that the fascia 31 is within the area of the window openings 7. The hollow needles are in their retracted positions in which they clear the window openings 7. Due to its intrinsic tension, tissue protrudes into the window openings 7. If this is desired, the tissue can be pulled further into the window openings 7 by generating an underpressure in the hollow space 6 of tube 1 (by means of an appropriate pumping device).

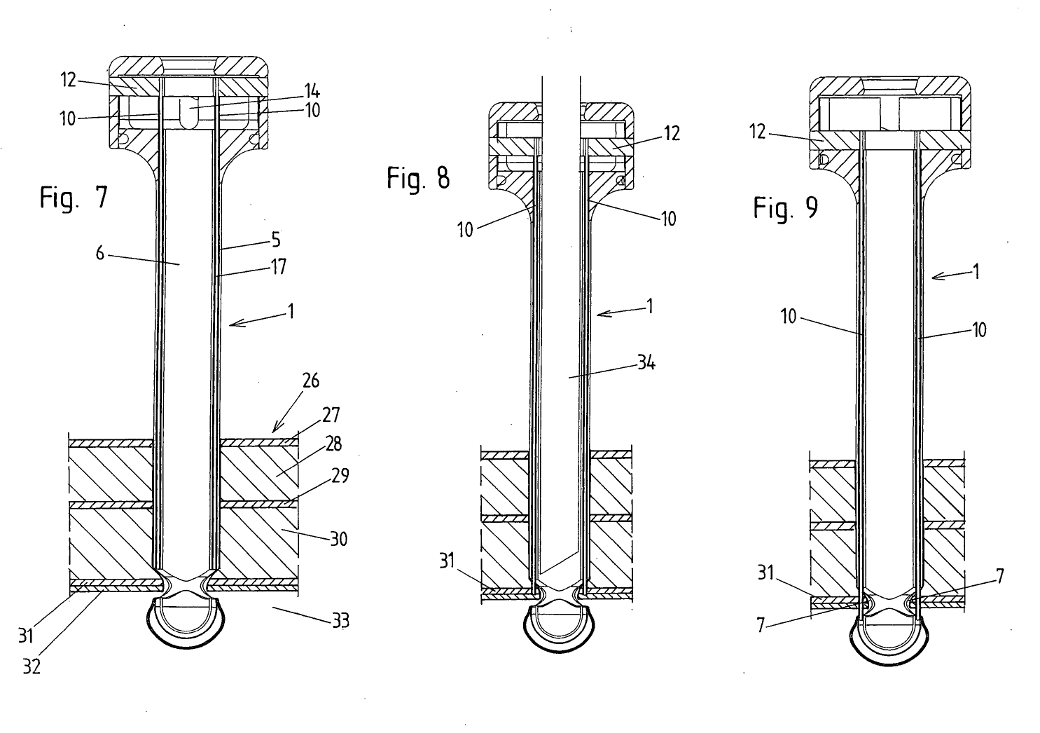

Next, the hollow needles 10 are slid forward. FIG. 8 shows an intermediate position. The tissue protruding into the window openings 7 is herein pierced by the hollow needles 10. This process can be visually checked through an endoscope 34 inserted into the inner hollow space 6 of tube 1, as is depicted schematically in FIG. 8.

FIG. 9 shows the state after the hollow needles 10 have been advanced into their end positions in which they cross the window openings 7. Together with the connecting section 18, the inner channels 17 of the hollow needles 10 form a continuous guide channel. FIG. 10 shows an enlarged detail of the depiction of FIG. 9.

Through this guide channel a threader element is subsequently inserted, as is shown in FIG. 11. At one end of the threader element 35 a suture thread 36 is fastened. The threader element 35, which is of greater length than the guide channel, can be formed for example by a flexible wire. The formation in the shape of a helical spring, whose diameter is less than the diameter of the guide channel, is also conceivable and possible. In the unbent state of the helical spring, the individual windings can be directly adjoining one another.

The thickness of the threader element 35 is greater than the width of the section of groove 19 extending between the steps 20, 21. The threader element 35, consequently, cannot leave the connecting section 18 but rather, when it exits from the front end of the inner channel of the one hollow needle 10, is guided by the curved guide channel until it enters again into the inner channel of the other hollow needle 10.

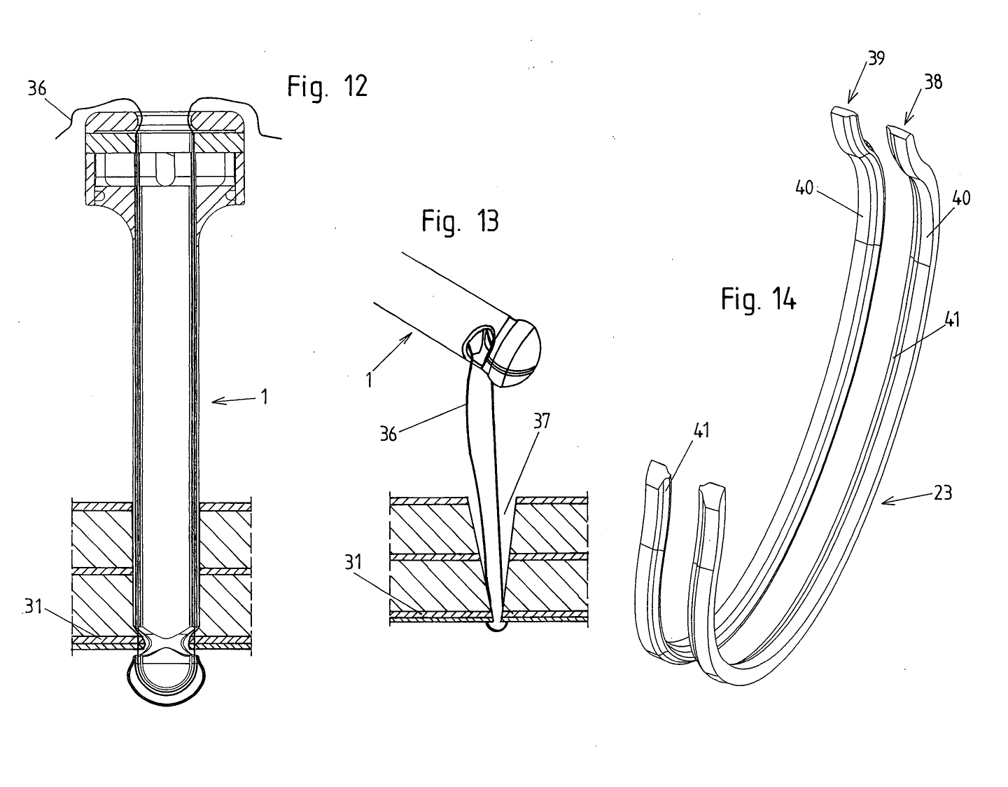

The threader element 35 is subsequently pulled out of the guide channel with the other end, whereby the thread 36 is threaded through the guide channel, as is evident in FIG. 12.

The hollow needles 10 are subsequently again retracted into their positions in which they clear the window openings 7, and the tube 1 is pulled out of the puncture channel 37. In FIG. 13 the puncture channel 37 is only for the sake of clarity shown in the open state. In fact, it closes after the tube 1 has been pulled out.

When the tube 1 is pulled out of the puncture channel, the thread 36, which penetrates the fascia 31 (and a section of the adjoining muscle layer 30) on both sides of the puncture channel, is retained by this body tissue and the segment of thread 36, which previously was located in the connecting section 18, is released out of the connecting section 18 and, by being guided through the narrower section of groove 19, located between the shoulders [sic: steps] 20, 21, and after tearing the closure device 23, arrives through its thinner region 25 in the outer space located outside of tube 1. The thread is furthermore pulled forwardly out of the inner channels 17 of the hollow needles.

This is shown in FIG. 13. The suture thread is thus placed in position and the thread ends can now be tied to complete the suture.

A further possible implementation of the closure device is shown in FIG. 14. Here the closure device is from the outset developed in two parts and includes two strip sections 38, 39, each of which has a thicker region 40, extending over the length of the strip section 38, 39, and a thinner projecting web 41. When the closure device is set into the groove 19, where a particular strip section 38, 39 is adhered on one of the steps 20, the free frontal faces of the webs 41 are adjacent and in contact on one another over the length of groove 19. In this closure device the pull-out force for thread 36 is decreased.

It would also be conceivable and possible that the connecting section 18 of the guide channel remains open. The narrower section of groove 19 adjoining the connecting section 18 of the guide channel toward the outer surface of the tube could in this case extend up to the outer surface of tube 1 (i.e. the step 20 and the outwardly adjoining wider section of groove 19 would be omitted). However, a closure device for the connecting section of the guide channel is preferably provided. It would also be conceivable and possible to implement this closure device in the form of a mechanical, openable and closable closure. For example a part, displaceable in the direction transversely to the connecting section, could be supported on or in the tube, such that it is displaceable by an actuating means between a position covering the connecting section and one clearing the connecting section.

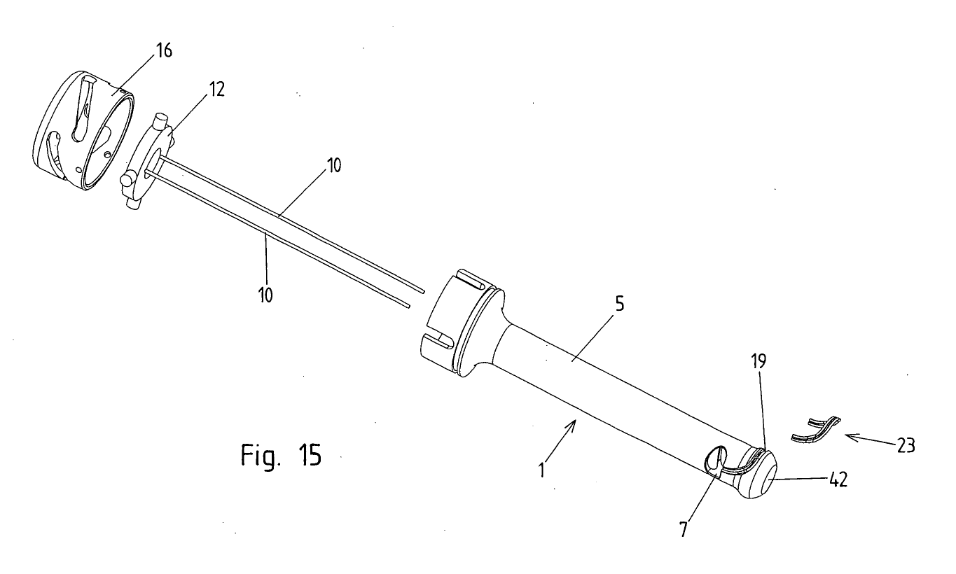

A further embodiment of an instrument according to the invention is depicted in FIG. 15 to 17. Apart from the modifications described in the following, this embodiment corresponds to the embodiment already described in conjunction with FIG. 1 to 13 and analogous parts were provided with the same reference numbers.

In contrast to the embodiment according to FIG. 1 to 13, the tube (=sleeve) 1 has at its front end an opening 42. To form the connecting section 18 of the guide channel, again a groove 19 is formed in the outer surface of tube 1, which now, however, extends over the shell wall of tube 1, such that the connecting section 18 extends within the shell wall 5 of tube 1. Starting from the window openings 7, the connecting section 18 has sections extending forwardly in the direction toward the insertion end 2, which transition in the form of arcs into a section connecting these two forwardly extending sections and extending in the circumferential direction.

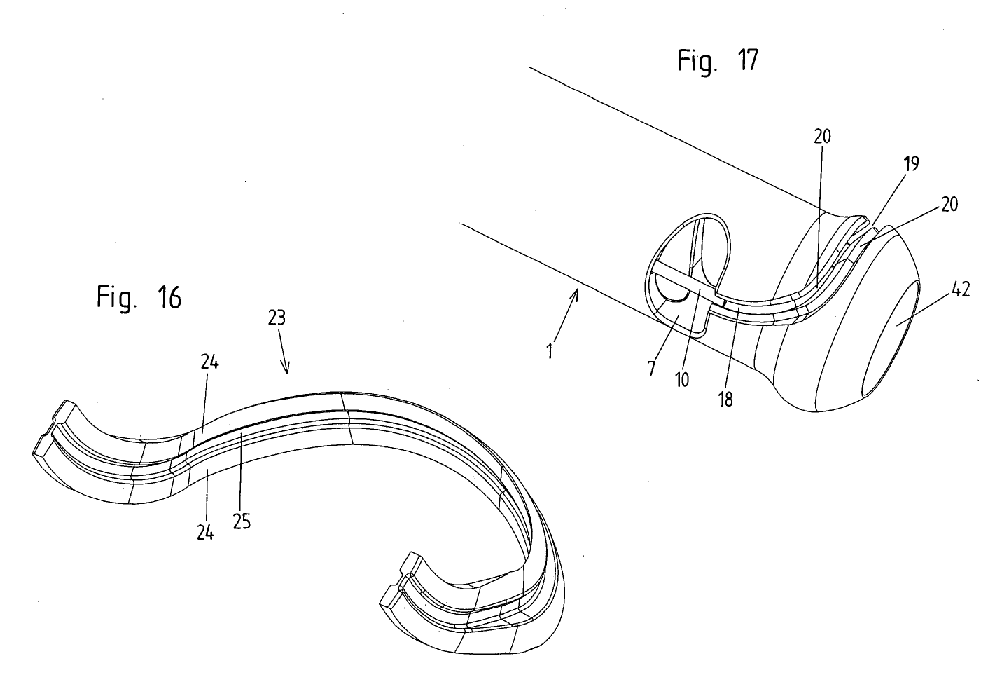

The closure device 23 has a curved shape adapted to this course of the connecting section 18 or the groove 19, as is depicted in FIG. 16. Again, between thicker regions 24 extending over the length of the closure device, is located a middle thinner region 25, which represents a rated break point for tearing [by] the thread 36. The closure device could also be implemented in two parts analogously to the closure device depicted in FIG. 14.

Due to the front opening 42 of tube 1, which continues the inner hollow space 6, the instrument can simultaneously be developed as a trocar. Further structural trocar parts required for this purpose, such as valves and/or sealings, are not shown in FIG. 15 for the sake of clarity. Such structural trocar parts are known and do not need to be further explained in this document.

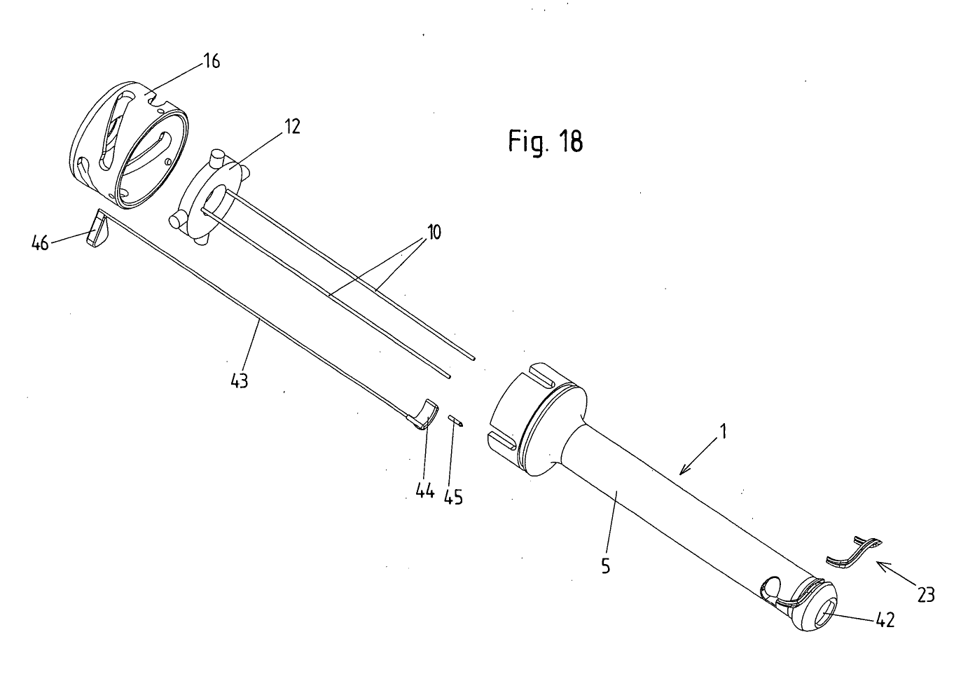

The embodiment depicted in FIG. 18 to 20 corresponds to the embodiment shown in FIG. 15 to 17, with the exception of the following modifications:

In the shell wall 5 of tube 1 is placed a further bore extending in the longitudinal direction, which is penetrated by a shaft 43. At the front end of the shaft a holder 44 for a cutting tool 45 is located. At the rearward end an actuating part 46 is disposed for rotating the shaft 43 and swivelling the holder 44.

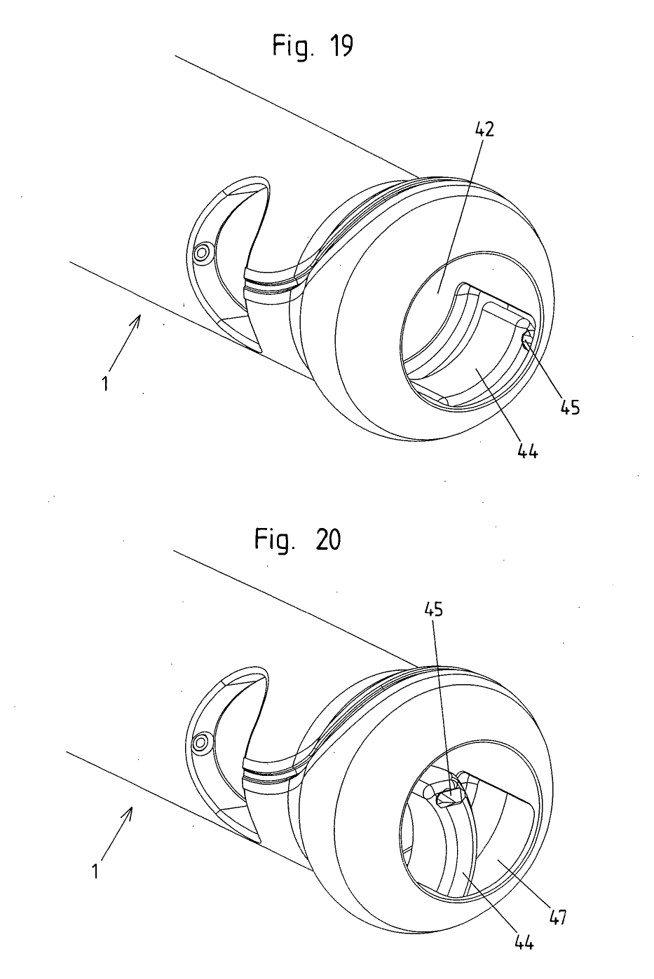

In the basic position the holder 44 with the cutting tool 45 is located in a recess 47 in the inner shell surface of tube 1, as is shown in FIG. 19. By rotating the shaft 43 the holder 44 with the cutting tool 45 is swivelled out of the recess 47, as is shown in FIG. 20. The cutting tool 45 is herein located near the insertion end 22 of tube 1 in the proximity of the opening 42. When the holder 44 in this position is swivelled back and forth by rotating the shaft back and forth while the instrument is being pressed onto the abdominal wall of the patient, a puncture channel for the tube 1 can be formed in order to introduce it through the tissue layers into the abdominal cavity.

The visual monitoring of the placement of the tube in the puncture channel and of the piercing of the posterior fascia by means of the hollow needles can each take place through an endoscope introduced into the hollow space 6 of the tube.

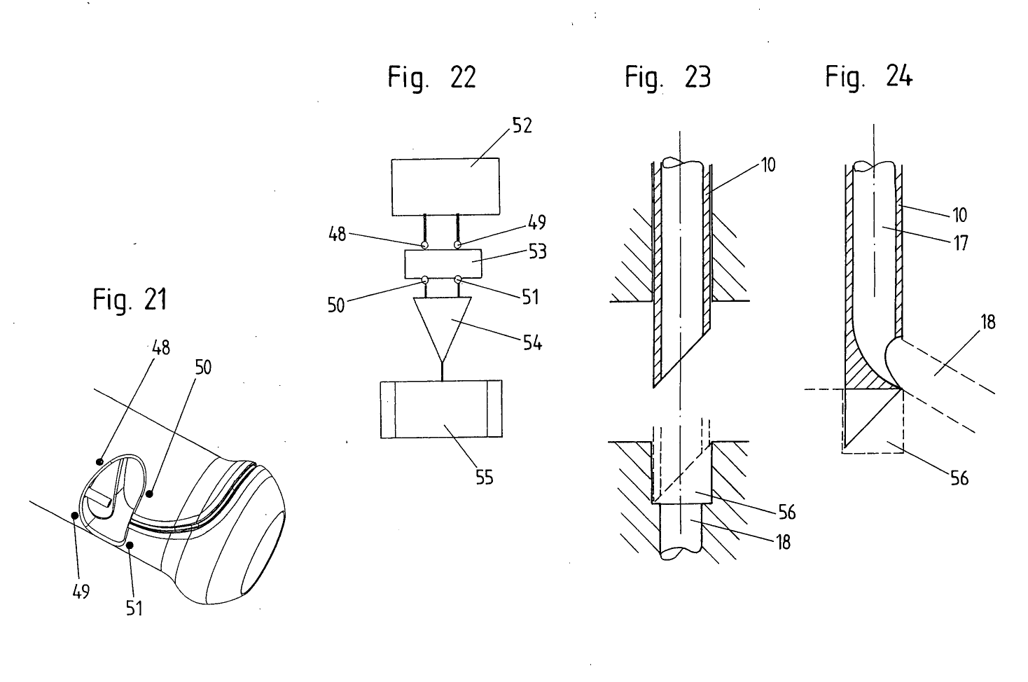

In addition, in a further embodiment of the invention shown schematically in FIGS. 21 and 22, monitoring of the placement of the tube can take place by means of impedance measurements. For this purpose electrodes 48 to 51 are disposed on the shell wall of the tube. Via two electrodes 49, 50 disposed at a spacing behind one of the window openings 7 a measuring pulse of constant current is impressed onto the tissue and the resulting voltage drop is measured with two electrodes 50, 51 disposed at a spacing in front of this window opening 7. This makes possible the determination of the tissue impedance, whereby a classification of the type of tissue in contact on the electrodes can be carried out. After the identification of the tissue, the correct puncture position can be indicated to the surgeon for example via an optical display, whereby this technique represents a supplement or an alternative to the visual check when applying the suture. FIG. 22 shows a block diagram of the configuration. The current flow between the electrodes 48, 49 is generated by the AC current source 52. The tissue 53 in between is shown schematically. The voltage between the electrodes 50, 51 is amplified by means of an amplifier 54 and supplied to an analysis and display unit 55.

FIG. 23 shows a schematic depiction of a feasible implementation of a countersupport for a particular hollow needle 10, which can be for example slanted at its front end. On the side opposite the exit site of the hollow needle 10 from the bore 8, a recess 56 is disposed, which advantageously is formed with an inlet slant (=inlet funnel). When the hollow needle 10 is advanced into its front end position, the front end of the hollow needle 10 moves into the recess 56 and is thereby centered. From the bottom of recess 56 extends the connecting section 18, preferably substantially aligned with the inner channel of hollow needle 10, while the recess 56 is comparatively wider.

Through the preferably provided slanted end of the hollow needle, an inwardly directed force component is generated during the piercing of the tissue, whereby the tissue is pressed further into the window opening 7. Such a slanted end of the hollow needle is also preferred for the other described embodiment examples (even though it is not depicted in FIG. 1 to 19).

In the embodiment of the hollow needle 10 depicted schematically in FIG. 24, the inner channel 17 of the hollow needle exits in the proximity of the tip of the hollow needle and laterally to it. The connecting section 18, indicated only in dashed lines in FIG. 24, is appropriately adapted in terms of placement and orientation. The tip of the hollow needle is preferably again slanted and, in the advanced end position of the hollow needle, is received in the pocket hole-like recess 56, which forms a countersupport for the hollow needle 10.

In particular for closing large trocar punctures, for example for trocar sizes of 15 mm and more, to each window opening 7 could also be assigned two bores in the shell wall 5 terminating at its margin remote from the insertion end, in each of which is disposed a hollow needle displaceable in the longitudinal direction. In this case, further, two connecting sections 18 would need to be provided, which each connect the inner channels of two hollow needles 10, which, in their front end position cross the opposing window openings. In this way, two individual button sutures closing the posterior fascia could be applied. Herein all four hollow needles can preferably be displaced simultaneously in the longitudinal direction of tube 1 by one common displacement device.

Different further modifications of the depicted embodiment examples of the invention are conceivable and possible without leaving the scope of the invention. Implementations of the tube 1 are for example also conceivable and possible, in which the tube does not have a rearward section 9 with an increased diameter. The bores 8 would in this case extend up to the rearward end of the tube and exit here from the shell wall. In this case behind the tube a device for the longitudinal displacement of the hollow needles could be disposed, which, for example, is connected with a mounting for the tube.

As is evident in the above description the scope of the invention is not limited to the depicted embodiment examples, but rather should be determined with reference to the attached claims together with its full range of possible equivalents.

### LEGEND TO THE REFERENCE NUMBERS

1 tube
2 insertion end
3 rearward end
4 longitudinal axis
5 shell wall
6 hollow space
7 window opening
8 bore
9 section
10 hollow needle
11 slotted link guide
12 needle mounting annulus
13 slotted link peg
14 slot
15 slotted link
16 actuating annulus
17 inner channel
18 connecting section
19 groove
20 step
21 step
22 partitioning wall
23 closure device
24 thicker region
25 thinner region
26 abdominal wall
27 skin
28 fatty layer
29 fascia
30 muscle layer
31 posterior fascia
32 peritoneum
33 abdominal cavity
34 endoscope
35 threader element
36 suture thread
37 puncture channel
38 strip section
39 strip section
40 thicker region
41 web
42 opening
43 shaft
44 holder
45 cutting tool
46 actuating part
47 recess
48 electrode
49 electrode
50 electrode
51 electrode
52 AC current source
53 tissue
54 amplifier
55 analysis and display unit
56 recess

1. An instrument for the surgical closure of a trocar puncture through a posterior fascia introduced during a laparoscopic operation comprises
a tube (1) with a forward insertion end (2) and a rearward end (3), in whose shell wall (5) opposing window openings (7) are formed, in which terminates at its margins remote from the insertion end (2) of the tube (1) at least one bore (8) extending in the shell wall (5) in the longitudinal direction of the tube (1),
hollow needles (10), each disposed in one of the bores (8) and displaceable between a retracted position, in which they clear the particular window opening (7), and an end position, in which they cross the window opening (7), and each having an inner channel (17),
wherein each of the inner channels (17) of the hollow needles (10) forms a section of a guide channel for guiding through a suture thread (36) and this guide channel further includes at least one curved connecting section (18), which connects the front ends of the inner channels (17) of two hollow needles (10) when they are in their end positions and crossing opposing window openings (7), which over its entire longitudinal length is covered with respect to the outer space encompassing the tube (1) by an openable closure device (23) or is connected via a slot with the outer space encompassing the tube (1), wherein a segment of the suture thread (36) located in the connecting section (18) can be brought out of the connecting section (18) of the guide channel into the outer space after opening the closure device (23) or through the slot.

2. Instrument as claimed in claim 1, wherein the openable closure device (23) is formed by a strip which can be torn by the thread (36) or can be unilaterally torn from the tube (1).

3. Instrument as claimed in claim 1, wherein the window openings (7) in a front end section of the tube (1) adjoining the insertion end (2).

4. Instrument as claimed in claim 3, wherein the window openings (7) are disposed in the front fourth of the longitudinal extent of the tube (1).

5. Instrument as claimed in claim 1, wherein only two opposing window openings (7) are provided.

6. Instrument as claimed in claim 5, wherein each of the opposing window openings (7) extends at least over 30% of the circumference of the tube (1).

7. Instrument as claimed in claim 1, wherein the hollow needles (10) in their retracted positions, in which they clear the window openings (7), as well as also in their advanced positions are located over at least a portion of their longitudinal extents in the bores (8).

8. Instrument as claimed in claim 1, wherein the bores (10) (sic: 8) in the shell wall (5) lead out of the tube (1) at a step of the tube (1) directed toward the rearward end (3) of the tube (1), which step is adjoined by a rearward expanded section (9) of the tube (1).

9. Instrument as claimed in claim 1, wherein a displacement device is provided for the simultaneous longitudinal displacement of the hollow needles (10).

10. Instrument as claimed in claim 9, wherein the displacement device comprises a needle mounting annulus (12), on which are disposed the hollow needles (10) in the proximity of their rearward ends.

11. Instrument as claimed in claim 10, wherein the needle mounting annulus (12) is displaceable in the longitudinal direction of the tube (1) by rotating a rotatable actuating annulus (16).

12. Instrument as claimed in claim 11, wherein the needle mounting annulus (12) is located in the rearward expanded section (9) of the tube (1).

13. Instrument as claimed in claim 12, wherein for its displacement in the longitudinal direction of the tube (1) the needle mounting annulus (12) comprises outwardly projecting slotted link pegs (13), which penetrate the slots (14) extending in the axial direction of the tube (1) in the expanded section (9) of the tube (1) and project into slotted links (15) in the actuating annulus (16).

14. Instrument as claimed in claim 1, wherein the hollow needles (10) in their retracted positions, in which they clear the window openings (7), as well as also in their end positions, in which they cross the window openings (7), project from the rearward ends of the bores (8).

15. Instrument as claimed in claim 1, wherein the hollow needles (10) in the advanced end positions project with front end sections into the recesses (56) forming countersupports and extending from the margins of the window openings (7) remote from the rearward end (3) of the tube (1).

16. Instrument as claimed in claim 1, wherein the tube (1) is closed at its insertion end (2) by a front wall.

17. Instrument as claimed in claim 16, wherein the connecting section (18) of the guide channel extends through the wall of the tube (1) forming the forward frontal face of the tube (1).

18. Instrument as claimed in claim 1, wherein the tube (1) has an opening (42) at its insertion end (2).

19. Instrument as claimed in claim 18, wherein the connecting section extends in the shell wall (5) of the tube (1) in a region of the tube (1) between the window openings (7) and the insertion end (2) of the tube (1).

20. Instrument as claimed in claim 1, wherein on the tube (1) electrodes (48, 49, 50, 51) are disposed for carrying out impedance measurements.

21. Instrument as claimed in claim 1, wherein in the advanced end position of the hollow needles (10) the guide channel, before the opening of the closure device (23) covering the connecting section (18), is closed over its entire longitudinal extent with respect to the outer space encompassing the tube (1).

22. Instrument as claimed in claim 1, wherein the thread (36) can be threaded into the guide channel by means of a threader element (35) whose length is greater than that of the guide channel and which can be pushed through the guide channel and is herein guided by the guide channel.
